(19) United States
(12) Patent Application Publication
(10) Pub. No.: US 2022/0273261 A1
(43) Pub. Date: Sep. 1, 2022

(54) ULTRASOUND IMAGING SYSTEM AND METHOD FOR MULTI-PLANAR IMAGING

(71) Applicant: GE Precision Healthcare LLC, Wauwatosa, WI (US)

(72) Inventors: Erik Normann Steen, Moss (NO); Svein Arne Aase, Trondheim (NO)

(21) Appl. No.: 17/186,731

(22) Filed: Feb. 26, 2021

(51) Int. Cl. A61B 8/14; A61B 8/00; G06N 3/02

(52) U.S. Cl. CPC A61B 8/145; A61B 8/463; A61B 8/4245; A61B 8/54; G06N 3/02

(57) ABSTRACT

An ultrasound imaging system and method of multi-planar ultrasound imaging includes repetitively scanning both a main image plane and a reference image plane with an ultrasound probe while in a multi-planar imaging mode, wherein the reference image plane intersects the main image plane along a line and where the main image plane is repetitively scanned at a higher-resolution than the reference image plane. The ultrasound imaging system and method includes displaying a main real-time image of the main image plane and a reference real-time image of the reference image plane concurrently on a display device.

### FIELD OF THE INVENTION

This disclosure relates generally to a method and ultrasound imaging system for multi-planar imaging where a main image plane is repetitively scanned at a higher resolution than a reference image plane.

### BACKGROUND OF THE INVENTION

In diagnostic ultrasound imaging, multi-planar imaging modes typically involve the acquisition and display of real-time images representing two or more image planes. Each of the real-time images is generated by repeatedly scanning one of the image planes. Both biplane imaging and triplane imaging are examples of multi-planar imaging modes. Biplane imaging typically involves the acquisition of slice data representing two planes disposed at ninety degrees to each other. Triplane imaging typically involves the acquisition of slice data representing three planes. The three planes may intersect along a common axis.

For many ultrasound workflows, a clinician will use a multi-planar imaging mode in order to more accurately position one of the image planes. For example, in order to confirm the accurate placement of one of the image planes, the clinician will rely on real-time images acquired from one or more other image planes. For example, multi-planar imaging modes are commonly used for cardiology. For many cardiac workflows, it is desired to accurately obtain images from a standard view. One or more images of the standard view may then be used for clinical purposes such as to help diagnose a condition, identify one or more abnormalities, or to obtain standardized measurement for quantitative comparison purposes. It is oftentimes difficult to accurately identify if an image plane is accurate positioned based on only a single view of the image plane. In order to obtain increased accuracy and confidence in the placement of an image plane, the clinician may use a multi-planar imaging mode in order to obtain more feedback about the placement of the imaging planes with respect to a desired anatomical structure/s.

For example, many standard cardiac views are defined with respect to an apex of the heart. For views such as an apical long axis view, apical four-chamber view, and a apical two-chamber view, it is necessary to position the image plane so it passes through the apex of the heart. If an image plane for an apical view does not pass the apex, the result may be a foreshortened view. In order to confirm that a view is correct, the clinician may rely on information obtained from other image planes in the multi-planar acquisition. For example, when following a workflow that requires an apical view, the clinician may use images obtained from the other image planes to position the ultrasound probe 106 so the main image plane passes through the apex of the heart.

One problem with using conventional multi-planar imaging modes is that the acquisition of more than one image plane has the potential to significantly degrade the image resolution compared to the acquisition of a single plane. For example, conventional multi-planar modes acquire ultrasound data of the same resolution from each of the image planes. The additional time to transmit and receive ultrasonic signals from the additional image planes decreases the relatively amount of time available for scanning each individual image plane. For example, the temporal resolution and/or the spatial resolution may be reduced in a multi-planar acquisition compared to a single-plane acquisition. For many workflows, the clinician is intending to only use the image from a main image plane for diagnostic purposes; images from the other one or more image planes are only used to guide the positioning of the main image plane.

Therefore, for these and other reasons, an improved system and method for multi-planar imaging is desired.

### BRIEF DESCRIPTION OF THE INVENTION

The above-mentioned shortcomings, disadvantages and problems are addressed herein which will be understood by reading and understanding the following specification.

In an embodiment, a method of multi-planar imaging includes repetitively scanning both a main image plane and a reference image plane with an ultrasound probe while in a multi-planar imaging mode. The reference image plane intersects the main image plane along a line. The main image is repetitively scanned at a higher resolution than the reference image plane. The method includes displaying a main real-time image of the main image plane and a reference real-time image of the reference image plane concurrently on a display device based on the repetitively scanning the main image plane and the reference image plane.

In another embodiment, an ultrasound imaging system includes an ultrasound probe, a display device, and a processor. The processor is configured to control the ultrasound probe to repetitively scan both a main image plane and a reference image plane with the ultrasound probe while in a multi-planar imaging mode. The reference image plane intersects the main image plane along a line, and the main image plane is repetitively scanned at a higher resolution than the reference image plane. The processor is configured to display a main real-time image of the main image plane and a reference real-time image of the reference image plane concurrently on the display device.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a representation of an ultrasound probe and two image planes in accordance with an embodiment;

FIG. 3 is a representation of an ultrasound probe and three image planes in accordance with an embodiment;

FIG. 4 is a representative of a screenshot in accordance with an embodiment;

FIG. 5 is a representation of a screenshot in accordance with an embodiment;

FIG. 6 is a schematic diagram of a neural network in accordance with an embodiment;

FIG. 7 is a schematic diagram showing input and output connections for a neuron of a neural network in accordance with an exemplary embodiment; and

FIG. 8 is a representation of a screenshot in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

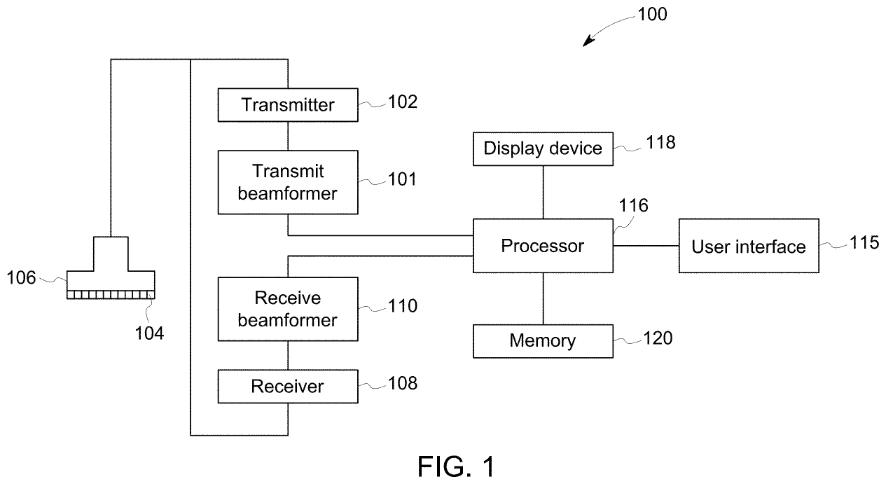

FIG. 1 is a schematic diagram of an ultrasound imaging system 100 in accordance with an embodiment. The ultrasound imaging system 100 includes a transmit beamformer 101 and a transmitter 102 that drive elements 104 within an ultrasound probe 106 to emit pulsed ultrasonic signals into a body (not shown) through one or more transmit events. The ultrasound probe 106 may be any type of ultrasound probe capable of a multi-planar acquisition mode. For example, the ultrasound probe 106 may be a dedicated bi-plane or tri-plane probe, or a 2D matrix array probe capable of 3D or 4D scanning. Still referring to FIG. 1, the pulsed ultrasonic signals are back-scattered from structures in the body, like blood cells or muscular tissue, to produce echoes that return to the elements 104. The echoes are converted into electrical signals by the elements 104 and the electrical signals are received by a receiver 108. The electrical signals representing the received echoes are passed through a receive beamformer 110 that outputs ultrasound data. According to some embodiments, the probe 106 may contain electronic circuitry to do all or part of the transmit beamforming and/or the receive beamforming. For example, all or part of the transmit beamformer 101, the transmitter 102, the receiver 108 and the receive beamformer 110 may be situated within the ultrasound probe 106. The terms “scan” or “scanning” may also be used in this disclosure to refer to acquiring data through the process of transmitting and receiving ultrasonic signals. The terms “data” and “ultrasound data” may be used in this disclosure to refer to either one or more datasets acquired with an ultrasound imaging system. A user interface 115 may be used to control operation of the ultrasound imaging system 100. The user interface 115 may be used to control the input of patient data, or to select various modes, operations, parameters, and the like. The user interface 115 may include one or more user input devices such as a keyboard, hard keys, a touch pad, a touch screen, a track ball, rotary controls, sliders, soft keys, or any other user input devices.

The ultrasound imaging system 100 also includes a processor 116 to control the transmit beamformer 101, the transmitter 102, the receiver 108 and the receive beamformer 110. The user interface 115 is in electronic communication with the processor 116. The processor 116 may include one or more central processing units (CPUs), one or more microprocessors, one or more microcontrollers, one or more graphics processing units (GPUs), one or more digital signal processors (DSP), and the like. According to some embodiments, the processor 116 may include one or more GPUs, where some or all of the one or more GPUs include a tensor processing unit (TPU). According to embodiments, the processor 116 may include a field-programmable gate array (FPGA), or any other type of hardware capable of carrying out processing functions. The processor 116 may be an integrated component or it may be distributed across various locations. For example, according to an embodiment, processing functions associated with the processor 116 may be split between two or more processors based on the type of operation. For example, embodiments may include a first processor configured to perform a first set of operations and a second, separate processor to perform a second set of operations. According to embodiments, one of the first processor and the second processor may be configured to implement a neural network. The processor 116 may be configured to execute instructions accessed from a memory. According to an embodiment, the processor 116 is in electronic communication with the ultrasound probe 106, the receiver 108, the receive beamformer 110, the transmit beamformer 101, and the transmitter 102. For purposes of this disclosure, the term “electronic communication” may be defined to include both wired and wireless connections. The processor 116 may control the ultrasound probe 106 to acquire ultrasound data. The processor 116 controls which of the elements 104 are active and the shape of a beam emitted from the ultrasound probe 106. The processor 116 is also in electronic communication with a display device 118, and the processor 116 may process the ultrasound data into images for display on the display device 118. According to embodiments, the processor 116 may also include a complex demodulator (not shown) that demodulates the RF data and generates raw data. In another embodiment the demodulation can be carried out earlier in the processing chain. The processor 116 may be adapted to perform one or more processing operations according to a plurality of selectable ultrasound modalities on the data. The data may be processed in real-time during a scanning session as the echo signals are received. The processor 116 may be configured to scan-convert the ultrasound data acquired with the ultrasound probe 106 so it may be displayed on the display device 118. Displaying ultrasound data in real-time may involve displaying the ultrasound data without any intentional delay. For example, the processor 116 may display each updated image frame as soon as each updated image frame of ultrasound data has been acquired and processed for display during the display of a real-time image. Real-time frame rates may vary based on the size of the region or volume from which data is acquired and the specific parameters used during the acquisition. According to other embodiments, the data may be stored temporarily in a buffer (not shown) during a scanning session and processed in less than real-time. According to embodiments that include a software beamformer, the functions associated with the transmit beamformer 101 and/or the receive beamformer 108 may be performed by the processor 116.

According to an embodiment, the ultrasound imaging system 100 may continuously acquire ultrasound data at a frame-rate of, for example, 10 Hz to 30 Hz. Images generated from the data may be refreshed at a similar frame-rate. Other embodiments may acquire and display data at different rates. For example, some embodiments may acquire ultrasound data at a frame rate of less than 10 Hz or greater than 30 Hz depending the size of each frame of data and the parameters associated with the specific application. For example, many applications involve acquiring ultrasound data at a frame rate of about 50 Hz. A memory 120 is included for storing processed frames of acquired data. In an exemplary embodiment, the memory 120 is of sufficient capacity to store frames of ultrasound data acquired over a period of time at least several seconds in length. The frames of data are stored in a manner to facilitate retrieval thereof according to its order or time of acquisition. The memory 120 may comprise any known data storage medium.

In various embodiments of the present invention, data may be processed by other or different mode-related modules by the processor 116 (e.g., B-mode, color Doppler, M-mode, color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate and combinations thereof, and the like. The image beams and/or frames are stored, and timing information indicating a time at which the data was acquired in memory may be recorded. The modules may include, for example, a scan conversion module to perform scan conversion operations to convert the image frames from beam space coordinates to display space coordinates. A video processor module may be provided that reads the image frames from a memory, such as the memory 120, and displays the image frames in real time while a procedure is being carried out on a patient. The video processor module may store the image frames in an image memory, from which the images are read and displayed.

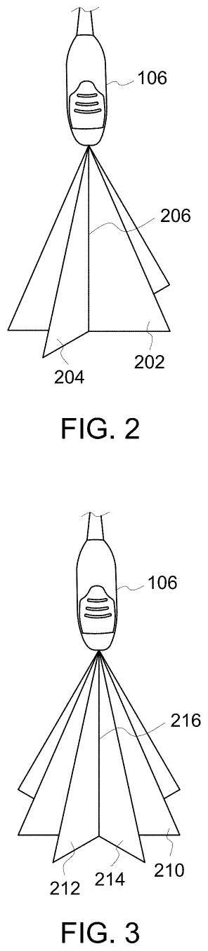

FIG. 2 is a schematic representation of the ultrasound probe 106 in a bi-plane imaging mode in accordance with an exemplary embodiment. The ultrasound probe 106 scans a main image plane 202 and a reference image plane 204 in the bi-plane imaging mode. The main image plane 202 intersects the reference image plane 204 other along a line 206. According to the embodiment shown in FIG. 2, the main image plane 202 may be orientated at a 90-degree angle with respect to the reference image plane 204. A bi-plane imaging mode, such as that shown in FIG. 2, is an example of a multi-planar imaging mode. In other embodiments, the main image plane 202 may be oriented at a different angle with respect to the reference image plane 204.

FIG. 3 is a schematic representation of the ultrasound probe 106 in a tri-plane imaging mode in accordance with an exemplary embodiment. The ultrasound probe 106 scans a main image plane 210, a first reference image plane 212, and a second reference plane 214 in the tri-plane imaging mode. The main image plane 210, the first reference image plane 212, and the second reference image plane 214 all intersect each other along a line 216. According to the embodiment shown in FIG. 3, the main image plane 210, the first reference image plane 212, and the second reference image plane 214 are all disposed at an angle of 60 degrees with respect to each other about the line 216. However, it should be appreciated that in other embodiments, the three image planes may be oriented at different angle with respect to each other while in a tri-plane imaging mode.

Both the bi-plane imaging mode schematically represented in FIG. 2 and the tri-plane imaging mode schematically represented in FIG. 3 are examples of multi-planar imaging modes. However, it is anticipated that multi-planar imaging modes in other embodiments may include a different number of image planes and/or the image planes in multi-planar imaging modes may be distributed in a different orientation with respect to each other and the ultrasound probe 106.

According to an embodiment, the processor 116 may be configured to enter a multi-planar imaging mode such as the bi-plane imaging mode represented in FIG. 2 or the tri-plane imaging mode represented in FIG. 3. The processor 116 may enter the multi-planar imaging mode in response to an input entered through the user interface 115, such as, for example, by receiving an input either directly selecting the multi-planar imaging mode or by receiving an input selecting a protocol or workflow that uses a multi-planar imaging mode as a default. According to other embodiments, the processor 116 may automatically enter the multi-planar imaging mode based on a selected protocol or workflow. A first exemplary embodiment will be described where the multi-planar imaging mode is a bi-plane imaging mode and will be described with respect to FIG. 2.

After entering the multi-planar mode, the processor 116 designates a main image plane, such as the main image plane 202, and at least one reference image plane, such as the reference plane 204. As will be described hereinafter, it is intended that a clinician will position the ultrasound probe 106 to acquire one or more clinically desired views from the main image plane 202 and will use the reference image plane 204 to help position or to confirm a position of the main image plane 202. The processor 116 is configured to control the ultrasound probe 106 to repetitively scan both the main image plane 202 and the reference image plane 204.

When displaying a real-time image of the main image plane 202, the processor 116 may, for instance, generate and display an image frame of the main image plane 202 each time that the main image plane 202 has been scanned. As described previously, an image plane is considered to have been “scanned” each time a frame of ultrasound data has been acquired from that particular image plane. The image frame displayed on the display device 118 represents the ultrasound data of the main image plane 202 acquired from the most recent scanning of the main image plane 202. For example, the processor 116 may display a main real-time image by generating and displaying a first image frame of the main image plane 202 the first time the main image plane has been scanned, generating and displaying a second image frame of the main image plane 202 the second time the main image plane 202 has been scanned, generating and displaying a third image frame of the main image plane 202 the third time the main image plane 202 has been scanned, etc.

Likewise, when displaying a real-time image of the reference image plane 204, the processor 116 may generate and display an image frame of the reference plane 204 each time the reference image plane 204 has been scanned. For example, the processor 116 may display a reference real-time image by generating and displaying a first image frame of the reference image plane 204 the first time the reference image plane 204 has been scanned, generating and displaying a second image frame of the reference image plane 204 the second time the reference image plane 204 has been scanned, generating and displaying a third image frame of the reference image plane 204 the third time the reference image plane has been scanned, etc.

While scanning a frame of ultrasound data, the processor 116 controls the transmit beamformer 101 and the transmitter 102 to emit a number of transmit events. Each transmit events may be either focused to a specific depth or unfocused. The number of transmit events is normally directly correlated to a spatial resolution of the resulting ultrasound data. Spatial resolution refers to the minimum distance at which two points may be discernable as separate objects. As a general rule, ultrasound data with higher spatial resolution permits the visualization of smaller structures than ultrasound data with a lower spatial resolution. For example, scanning the main image plane 202 while using a higher number of transmit events will usually result in higher spatial resolution ultrasound data than scanning the main image plane 202 while using a reduced number of transmit events if the other acquisition parameters remain the same. Higher spatial resolution ultrasound data enables the processor 116 to display an image frame or a real-time image with a higher spatial resolution than would be possible using lower spatial resolution ultrasound data.

Each transmit event takes time for the pulsed ultrasonic signals to penetrate into the tissue being examined and time for back-scattered signals and/or the reflected signals generated in response to each transmit event to travel from the originating depth in the tissue back to the ultrasound probe 106. Since both the pulsed ultrasonic signals emitted from the ultrasound probe 106 during each transmit event and the backscattered and/or reflected signals generated in response to the transmit events are limited by the speed of sound, acquiring a frame of data using a higher number of transmit events takes more time than acquiring the frame of data using fewer transmit events if all the other parameters remain constant. As a consequence, it typically takes more time to acquire each frame of higher spatial resolution ultrasound data compared to the time it takes to acquire each frame of lower spatial resolution ultrasound data if all the other parameters remain constant.

As a result of the inverse relationship between spatial resolution and temporal resolution, or the frame-rate, it is typically necessary to trade-off spatial resolution to increase temporal resolution and vice versa. For applications, such as cardiology, where it is desirable to have both high temporal resolution (i.e., frame-rate) and a high spatial resolution, multi-planar modes pose a particular challenge. Instead of just acquiring ultrasound data by scanning a single image plane, multi-planar imaging modes acquire ultrasound data by scanning two or more image planes. As was described in the Background of the Invention section, conventional multi-planar imaging modes scan the two or more image planes with the same resolution. As a result, in conventional multi-planar imaging modes, the resolution of each of the planes is oftentimes lower that would be optimal, especially for applications requiring both high spatial resolution and high temporal resolution.

The processor 116 may be configured to repetitively scan both the main image plane 202 and the reference image plane 204. The processor 116 may be configured to repetitively scan the main image plane 202 at a higher resolution than the reference image plane 204.

According to an embodiment, the processor 116 may be configured to repetitively scan the main image plane 202 and the reference image plane 204 at two different frame rates. For example, the processor 116 may be configured to repetitively scan the main image plane 202 at a higher temporal resolution than the reference image plane 204. The processor 116 is configured to display a main real-time image of the main image plane 202 on the display device 118 while concurrently displaying a reference real-time image of the reference image plane 204 on the display device 118. Since the main image plane 202 was repetitively scanned at a higher temporal resolution than the reference image plane 204, the temporal resolution of the main real-time image will also be higher than the temporal resolution of the reference real-time image. In other words, the main real-time image will have a higher frame-rate than the reference image.

According to an embodiment, the processor 116 may be configured to repetitively scan the main image plane 202 and the reference image plane 204 at two different spatial resolutions. For example, the processor 116 may be configured to repetitively scan the main image plane 202 at a higher spatial resolution than the reference image plane 204. For example, the processor 116 may use a higher number of transmit events to acquire each frame of ultrasound data from the main image plane 202 compared to the reference image plane 204. The processor 116 is configured to display a main real-time image of the main image plane 202 on the display device 118 while concurrently displaying a reference real-time image of the reference image 204 on the display device 118. Since the main image plane 202 was repetitively scanned at a higher spatial resolution than the reference image plane 204, the spatial resolution of the main real-time image will also be higher than the spatial resolution of the reference real-time image.

According to an embodiment, the processor 116 may be configured to repetitively scan the main image plane 202 at both a spatial resolution and a temporal resolution that is different from that at which the reference image plane 204 is repetitively scanned. For example, the processor 116 may be configured to repetitively scan the main image plane 202 at both a higher spatial resolution and a higher temporal resolution than the reference image plane 204. For example, the processor 116 may use a higher number of transmit events to acquire each frame of ultrasound data from the main image plane 202 compared to the reference image plane 204. The processor 116 may also acquires frames of ultrasound data of the main image plane 202 at a higher temporal resolution compared to the reference plane 204. The processor 116 is configured to display a main real-time image of the main image plane 202 while concurrently displaying a reference real-time image of the reference image plane 204. Since the main image plane 202 was repetitively scanned at both a higher spatial resolution and a higher temporal resolution than the reference image plane 204, the main real-time image of the main plane 202 will have both a higher spatial resolution and a higher temporal resolution than the reference real-time image of the reference plane 204.

According to an embodiment, the processor 116 may be configured to scan the reference image plane 204 to a shallower depth than the main image plane 202. For example, the processor 116 may only acquire ultrasound data from the reference image plane 204 to a first depth from the elements 104 of the probe 106. The processor 116 may be configured to acquired ultrasound data from the main image plane 202 to a deeper depth from the elements 104 of the probe 106. Acquiring ultrasound data by scanning the reference image plane 204 to a shallower depth than the main image plane 202 may be used to help reduce the overall time spent scanning the reference image plane 204, which, in turn, allows a greater percentage of time to be spent scanning the main image plane 202. Repetitively scanning the reference image plane 204 to a shallower depth may be used in combination with either one or both of repetitively scanning the main image frame 202 at a higher spatial resolution than the reference image plane 204 and repetitively scanning the main image plane 202 at a higher temporal resolution than the reference image plane 204 according to various embodiments.

By spending a relatively larger amount of time acquiring ultrasound data from the main image plane 202 than the reference image plane 204, the processor 116 is configured to scan the main image plane 202 at a higher resolution than the reference image plane 204. This in turn enables the processor 116 to display a main real-time image of the main image plane 202 with a higher resolution than the reference real-time image of the reference image plane 204. Additionally, by reducing the amount of time spent repetitively scanning the reference image plane 204, the processor 116 is able to display a main real-time image with a higher resolution than would be possible with a conventional system and technique that equally allocates scanning time between both the main image plane 202 and the reference image plane 204. The system and method described hereinabove is particularly advantageous for clinical applications where both a high spatial resolution and a high temporal resolution are valuable, such as cardiology.

According to an embodiment, the processor 116 may be configured to spend more time scanning a main image plane in a tri-plane imaging mode. For example, FIG. 3 includes a main image plane 210, a first reference image plane 212, and a second reference image plane 214. The processor 116 may be configured to repetitively scan the main image plane 210 at a higher resolution than either of the reference image planes. Each reference image plane may be scanned with one or both of a lower temporal resolution and a lower spatial resolution than the main image plane 210 in a manner similar to that which was described with respect to the reference image plane 202 of FIG. 2. The main real-time image of the main image plane 210 will therefore have a higher resolution (spatial resolution and/or temporal resolution) than a first reference real-time image of the first reference image plane 212 and a second reference real-time image of the second reference image plane 214. Those skilled in the art should appreciate that the method described hereinabove may also be applied to multi-planar imaging modes with more than 3 separate image planes.

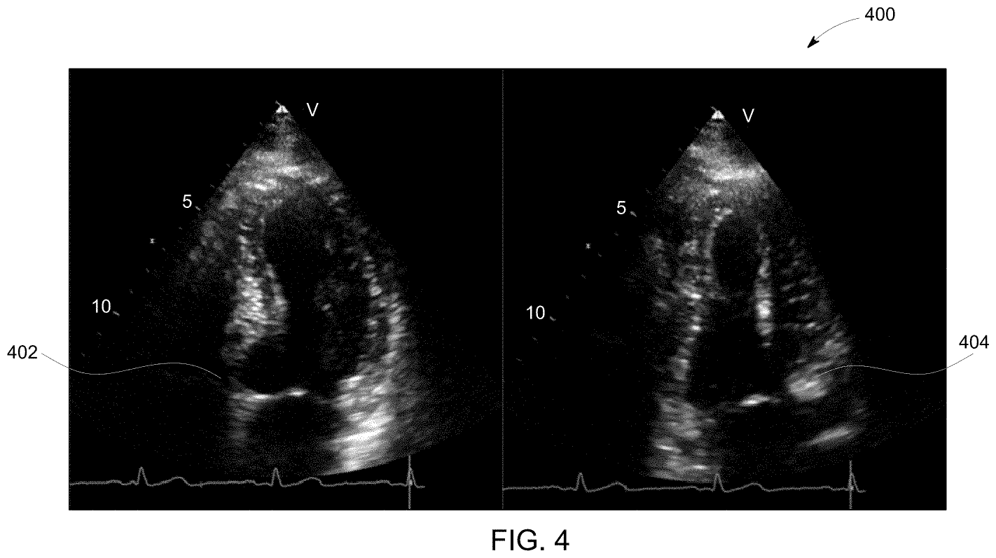

FIG. 4 is a screenshot 400 that may be displayed on the display device 118 according to an exemplary embodiment. The screenshot 400 includes a main image frame 402 and a reference image frame 404. The main image frame 402 shown in FIG. 4 may be a frame of a main real-time image and the reference image frame 404 may be a frame of a reference real-time image. Since the screenshot 400 represents a single point in time, only a single frame of the main real-time image and the reference real-time image are depicted. According to an embodiment, the main image frame 402 may be replaced by an updated main image frame after an additional frame of ultrasound data is acquired of the main image plane by the ultrasound probe 106. Likewise, the reference image frame 404 will be replaced by an updated reference image frame after an additional frame of ultrasound data is acquired of the reference image plane. The screenshot 400 shows the main image frame 402 and the reference image frame in a side-by-side format.

During the process of repetitively scanning both the main image plane 202 and the reference image plane 204, the side-by-side format, such as that shown in FIG. 4 allows that clinician to easily use the real-time reference image (represented by reference image frame 404) in order to position and orient the ultrasound probe 106 so that the main real-time image (represented by the main image frame 402) captures the desired standardized view plane or a target anatomical feature. As discussed hereinabove, the main real-time image (represented by the main image frame 402) is of a higher resolution than the reference real-time image (represented by reference image frame 404). The reference real-time image is not intended to be used for diagnostic purposes. Rather, the reference real-time image is intended to be used in order to properly position the main real-time image, which will be used to capture diagnostically useful images. As such, reducing the resolution of the reference real-time image enables the main-real time image to have a higher resolution compared to conventional techniques. Additionally, the side-by-side format, such as that shown in FIG. 4, allows the clinician to easily keep track of both the main real-time image (represented by the main image frame 402) and the reference real-time image (represented by reference image frame 404) while positioning the ultrasound probe 106 to image the desired anatomy of the patient.

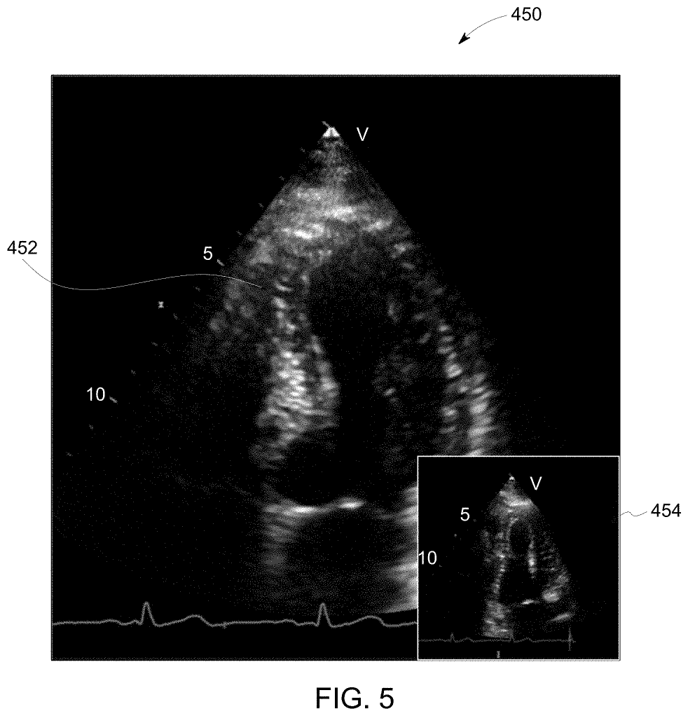

FIG. 5 is a screenshot 450 that may be displayed on the display device 118 according to an exemplary embodiment. The screenshot 450 includes a main image frame 452 and a reference image frame 454. The main image frame 452 shown in FIG. 4 may be a frame of a main real-time image and the reference image frame 454 may be a frame of a reference real-time image according to an embodiment. Since the screenshot 450 represents a single point in time, only a single frame of the main real-time image and only a single frame of the reference real-time image are depicted. According to an embodiment, the main image frame 452 will be replaced by an updated main image frame after an additional frame of ultrasound data is acquired of the main image plane by the ultrasound probe 106. Likewise, the reference image frame 454 will be replaced by an updated reference image frame after an additional frame of ultrasound data is acquired of the reference image plane. According to an embodiment, the main image plane may be the first image plane 202 and the reference image plane may be the second image plane 204 (shown in FIG. 2). The screenshot 450 shows the main image frame 452 and the reference image frame 454 in a picture-in-picture format since the reference image frame 454 is displayed as a region within the main image frame 452. According to an embodiment where the main image frame 452 is a frame of a main real-time image and the reference image frame 454 is a frame of a reference real-time image, FIG. 5 also shows a main real-time image (represented by main image frame 452) and a reference real-time image (represented by reference image frame 454) displayed in a picture-in-picture format.

The picture-in-picture format, such as that shown in FIG. 5, allows most of the available screen space to be used for displaying the main real-time image (represented by main image frame 452) while dedicating a much smaller amount of screen space to displaying the reference real-time image (represented by reference image frame 454). As such, the picture-in-picture format may be particularly advantageous for ultrasound imaging system where screen space is at a premium, such as portable, hand-held, or hand-carried ultrasound imaging systems. However, it should be appreciated that the picture-in-picture format may also be used by systems with larger screens such as cart-based systems, console-based systems, wall-mounted systems, ceiling-mounted systems, etc.

According to an embodiment, the processor 116 may be configured to automatically detect a target anatomical feature in either the main real-time image or the reference real-time image. The processor 116 may be configured to use image processing techniques such as edge detection, B-splines, shape-based detection algorithms, average intensity, segmentation, speckle tracking, or any other image-processing based techniques to identify one or more target anatomical features. According to other embodiments, the processor 116 may be configured to implement one or more neural networks in order to detect the target anatomical feature/s in the main real-time image or the reference real-time image. The one or more neural networks may include a convolutional neural network (CNN) or a plurality of convolutional neural networks according to various embodiments.

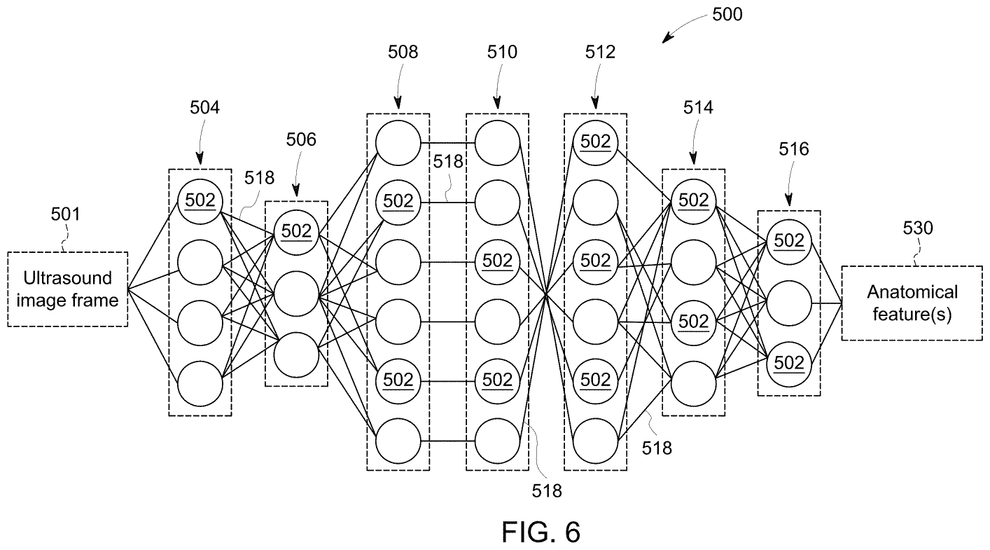

FIG. 6 depicts a schematic diagram of a neural network 500 having one or more nodes/neurons 502 which, in some embodiments, may be disposed into one or more layers 504, 506, 508, 510, 512, 514, and 516. Neural network 500 may be a deep neural network. As used herein with respect to neurons, the term “layer” refers to a collection of simulated neurons that have inputs and/or outputs connected in similar fashion to other collections of simulated neurons. Accordingly, as shown in FIG. 6, neurons 502 may be connected to each other via one or more connections 518 such that data may propagate from an input layer 504, through one or more intermediate layers 506, 508, 510, 512, and 514, to an output layer 516. The one or more intermediate layers 506, 508, 510, 512, and 514 are sometimes referred to as “hidden layers.”

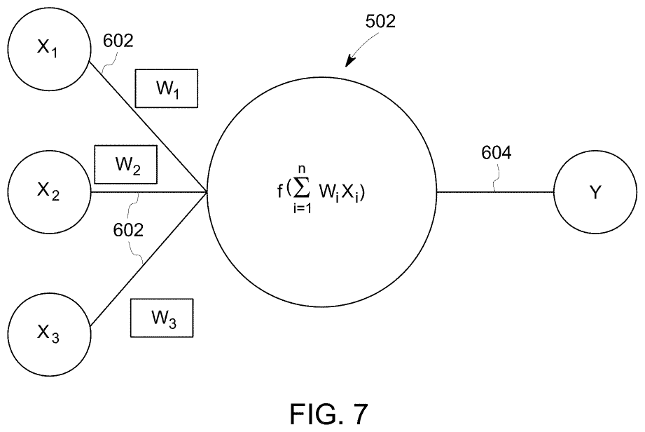

FIG. 7 shows input and output connections for a neuron in accordance with an exemplary embodiment. As shown in FIG. 7, connections (e.g., 518) of an individual neuron 502 may include one or more input connections 602 and one or more output connections 604. Each input connection 602 of neuron 502 may be an output connection of a preceding neuron, and each output connection 604 of neuron 502 may be an input connection of one or more subsequent neurons. While FIG. 7 depicts neuron 502 as having a single output connection 604, it should be understood that neurons may have multiple output connections that send/transmit/pass the same value. In some embodiments, neurons 502 may be data constructs (e.g., structures, instantiated class objects, matrices, etc.), and input connections may be received by neuron 502 as weighted numerical values (e.g., floating point or integer values). For example, as further shown in FIG. 7, input connections X1, X2, and X3 may be weighted by weights W1, W2, and W3, respectively, summed, and sent/transmitted/passed as output connection Y. As will be appreciated, the processing of an individual neuron 502 may be represented generally by the equation:

$$Y = f ( {\sum }_{i = 1}^{n} {W}_{i} {X}_{i} )$$

where n is the total number of input connections 602 to neuron 502. In one embodiment, the value of Y may be based at least in part on whether the summation of WiXi exceeds a threshold. For example, Y may have a value of zero (0) if the summation of the weighted inputs fails to exceed a desired threshold.

As will be further understood from FIGS. 6 and 7, input connections 602 of neurons 502 in input layer 504 may be mapped to an input 501, while output connections 604 of neurons 502 in output layer 516 may be mapped to an output 530. As used herein, “mapping” a given input connection 602 to input 501 refers to the manner by which input 501 affects/dictates the value said input connection 602. Similarly, as also used herein, “mapping” a given output connection 604 to output 530 refers to the manner by which the value of said output connection 604 affects/dictates output 530.

Accordingly, in some embodiments, the acquired/obtained input 501 is passed/fed to input layer 504 of neural network 500 and propagated through layers 504, 506, 508, 510, 512, 514, and 516 such that mapped output connections 604 of output layer 516 generate/correspond to output 530. As shown, input 501 may include one or more ultrasound image frames that are, for example, part of a main real-time image or a reference real-time image. The image may include one or more structures that are identifiable by the neural network 500. Further, output 530 may include structures, landmarks, contours, or planes associated with standard views.

Neural network 500 may be trained using a plurality of training datasets. According to various embodiments, the neural network 500 may be trained with a plurality of ultrasound images. The ultrasound images may include annotated ultrasound image frames with one or more annotated structures of interest in each of the ultrasound image frames. Based on the training datasets, the neural network 500 may learn to identify one or more anatomical structures from the volume data. The machine learning, or deep learning, therein (due to, for example, identifiable trends in placement, size, etc. of anatomical features) may cause weights (e.g., W1, W2, and/or W3) to change, input/output connections to change, or other adjustments to neural network 500. Further, as additional training datasets are employed, the machine learning may continue to adjust various parameters of the neural network 500 in response. As such, a sensitivity of the neural network 500 may be periodically increased, resulting in a greater accuracy of anatomical feature identification.

According to an embodiment, the neural network 500 may be trained to identify anatomical structures in the ultrasound image frames and/or ultrasound data. For example, according to an embodiment where the ultrasound data is cardiac data, the neural network 500 may be trained to identify a target anatomical feature such as the right ventricle, the left ventricle, the right atrium, the left atrium, one or more valves, such as the tricuspid value, the mitral valve, the aortic valve, the apex of the left ventricle, the septum, etc.

Once the target anatomical feature has been identified by the processor 116, the processor 116 may be configured to display a graphical indicator to mark the target anatomical feature in one of the main real-time image and/or the reference real-time image. The processor 116 may be configured to detect the position of the target anatomical feature in each frame of the main real-time image or in each frame of the reference real-time image and update the position of the graphical indicator for each image frame of the respective real-time image so that the graphical indicator represents a real-time position of the anatomical feature. In other embodiments, the processor 116 may be configured to detect the target anatomical feature in a single image frame. For example, the processor 116 may be configured to detect the target anatomical feature after the clinician has actuated a “freeze” command via the user interface 115 to display a single image frame of the main real-time image and a single frame of the reference real-time image.

The processor 116 may be configured to display a projection of the graphical indicator on the other of the main real-time image and the reference real-time image. For example, if the processor 116 detects the target anatomical feature in the main real-time image, the processor 116 would display a graphical indicator in the main real-time image to mark the target anatomical feature. In addition to displaying the graphical indicator, the processor 116 may be configured to display a projection of the graphical indicator on the reference real-time image.

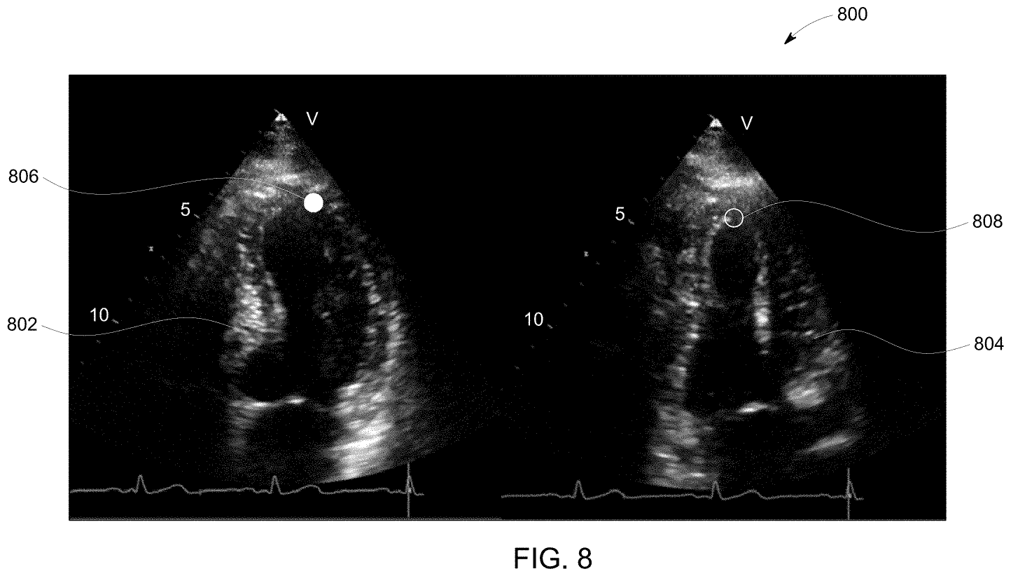

FIG. 8 is a screenshot 800 that may be displayed on the display device 118 in accordance with an embodiment. The screenshot 800 includes a main image frame 802 and a reference image frame 804 displayed in the side-by-side format described previously with respect to FIG. 4. The main image frame 802 shown in FIG. 8 may be a frame of a main real-time image and the reference image frame 804 may be a frame of a reference real-time image. Since the screenshot 800 represents a single point in time, only a single frame of the main real-time image and a single frame of the reference real-time image are depicted. The screenshot 800 includes a graphical indicator 806, which is shown on the main image frame 802. According to other embodiments, a graphical indicator may be shown on the reference image frame 804 instead of or in addition to the displaying the graphical indicator 806 on the main image frame 802. The screenshot 800 also includes a projection of the graphical indicator 808 on the reference image frame 804. The graphical indicator 806 marks the location of the target anatomical structure that is shown in the main image frame 802. The projection of the graphical indicator 808 may be used to represent a projected position of the target anatomical structure onto the reference image frame 804. For example, in the screenshot 800, the projection of the graphical indicator 808 is shown as an outline of a circle while the graphical indicator 806 is shown as a solid circle. In the case of the embodiment shown in FIG. 8, the outline of the circle indicates that the target anatomical structure is not in the reference image plane represented by the reference image frame 804. The target anatomical structure is either in front of or behind the reference image plane.

The processor 116 may be configured to adjust the appearance of the projection of the graphical indicator 808 in order to indicate the position of the target anatomical structure with respect to the reference image 804. For example, the processor 116 may be configured to used different colors, intensities, or levels of fill to illustrate the relative position of the target anatomical structure with respect to the reference image plane 204. For example, in FIG. 8, the projection of the graphical indicator 808 is shown as an outline of a circle that is not filled-in in the center. According to an embodiment, the processor 116 may be configured to adjust an amount of fill used for the projection of the graphical indicator 808 as the target anatomical structure is closer to the reference image plane depicted by the reference image. The processor 116 may show the projection of the graphical indicator 808 as completely solid, in a manner similar to the graphical indicator 806, when the target anatomical structure is in the reference image plane. The processor 116 may likewise adjust an intensity of the projection of the graphical indicator 808 based on the relatively position of the target anatomical structure with respect to the reference image plane. For example, the projections of the graphical indicator 800 may be displayed at a maximum intensity when the target anatomical structure is positioned in the reference image plane and at progressively lower intensities as the distance between the target anatomical structure and the reference image plane increases. Other embodiments may use different graphical indicators to mark the location of the target anatomical structure. For example, the graphical indicator may be a different polygon, such as a square, a rectangle, a triangle, etc., the graphical indicator may be a cross or a plus, or the graphical indicator may be any other graphical technique used to mark a specific portion of the image.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of multi-planar ultrasound imaging comprising:
repetitively scanning both a main image plane and a reference image plane with an ultrasound probe while in a multi-planar imaging mode, where the reference image plane intersects the main image plane along a line, and where the main image plane is repetitively scanned at a higher resolution than the reference image plane; and
displaying a main real-time image of the main image plane and a reference real-time image of the reference image plane concurrently on a display device based on said repetitively scanning the main image plane and the reference image plane.

2. The method of claim 1, wherein said displaying the main real-time image of the main image plane and the reference real-time image of the reference plane comprises displaying the reference real-time image and the main real-time image in a side-by-side format.

3. The method of claim 1, wherein said displaying the main real-time image of the main image plane and the reference real-time image of the reference plane comprises displaying the reference real-time image and the main real-time image in a picture-within-picture format, where the reference real-time image is displayed within the main real-time image.

4. The method of claim 1, wherein the main image plane is scanned at a higher temporal resolution than the reference image plane.

5. The method of claim 1, wherein the main image plane is scanned at a higher spatial resolution than the reference image plane.

6. The method of claim 1, wherein the main image plane is scanned at both a higher spatial resolution and a higher temporal resolution than the reference image plane.

7. The method of claim 1, wherein said repetitively scanning both the main image plane and the reference image plane comprises repetitively scanning the reference image plane to a shallower depth than the main image plane.

8. The method of claim 1, further comprising automatically detecting with a processor, a target anatomical structure in one of the main real-time image or the reference real-time image and displaying a graphical indicator to mark the target anatomical structure in the one of the main real-time image or the reference real-time image.

9. The method of claim 8, further comprising automatically displaying, with the processor, a projection of the graphical indicator on the other of the main real-time image or the reference real-time image.

10. The method of claim 8, wherein said automatically detecting the target anatomical feature comprises implementing one or more neural networks with the processor.

11. The method of claim 8, wherein both the main image plane and the reference image plane pass through a heart, and wherein the target anatomical structure comprises an apex of the heart.

12. An ultrasound imaging system comprising:
an ultrasound probe;
a display device; and
a processor, wherein the processor is configured to:
control the ultrasound probe to repetitively scan both a main image plane and a reference image plane with the ultrasound probe while in a multi-planar imaging mode, where the reference image plane intersects the main image plane along a line, and where the main image plane is repetitively scanned at a higher resolution than the reference image plane; and
display a main real-time image of the main image plane and a reference real-time image of the reference image plane concurrently on the display device.

14. The ultrasound imaging system of claim 12, wherein the processor is configured to display the main real-time image of the main image plane and the reference real-time image of the reference plane in a side-by-side format.

15. The ultrasound imaging system of claim 12, wherein the processor is configured to display the main real-time image of the main image plane and the reference real-time image of the reference plane in a picture-in-picture format.

16. The ultrasound imaging system of claim 12, wherein the processor is configured to control the ultrasound probe to repetitively scan the main image plane at a higher temporal resolution than the reference image plane.

17. The ultrasound imaging system of claim 12, wherein the processor is configured to control the ultrasound probe to repetitively scan the main image plane at a higher spatial resolution than the reference image plane.

18. The ultrasound imaging system of claim 12, wherein the processor is configured to repetitively scan the reference image plane to a shallower depth than the main image plane.

19. The ultrasound imaging system of claim 12, wherein the processor is configured to automatically detect a target anatomical structure in at least one of the main real-time image and the reference real-time image, and wherein the processor is configured to automatically display a graphical indicator to mark the target anatomical structure on at least one of the main real-time image or the reference real-time image.

20. The ultrasound imaging system of claim 19, wherein the processor is configured to implement one or more neural networks in order to automatically detect the target anatomical structure in the at least one of the main real-time image and the reference real-time image.
